(19) United States
(12) Patent Application Publication
(10) Pub. No.: US 2008/0287532 A1
(43) Pub. Date: Nov. 20, 2008

(54) OXACYCLOPENTENE-2 DERIVATIVES

(76) Inventors: Joseph Schwarz, (US); Michael Weisspapir, (US)

Correspondence Address: ALPHARX INC., 168 KONRAD CRESCENT, SUITE 200, MARKHAM, L3R 9T9, CA

(21) Appl. No.: 11/750,925

(22) Filed: May 18, 2007

(51) Int. Cl. A61K 31/341; A61P 25/28; A61P 29/00; A61P 31/00; A61P 35/00; A61P 37/00; A61P 37/08; A61P 9/00; C07D 307/32

(52) U.S. Cl. 514/473; 549/479

(57) ABSTRACT

The present invention relates to oxacyclopentene-2 derivatives as compounds possessing antiinflammatory, neuroprotective, immunomodulatory and cardiovascular activities in mammals.

### FIELD OF THE INVENTION

The present invention relates to oxacyclopentene-2 derivatives as compounds possessing antiinflammatory, neuroprotective, immunomodulatory and cardiovascular activities in mammals.

### BACKGROUND OF THE INVENTION

Oxacyclopentene (oxalene, dihydrofuran) derivatives are used in organic chemistry as synthetic intermediates [1,2]; they are also mentioned as components of fragrances and flavors [3,4]. Several compounds containing oxacyclopentene moiety have been previously described as biologically active substances. Dihydrofuran structure may be part of some prostaglandins (PGs) molecules. Prostaglandins are naturally occurring cyclic 20-carbon fatty acids that are synthesized by various types of eukaryotic cells in response to external stimuli and play an important role in a variety of physiological responses. PGs were shown to act as microenvironmental hormones and intracellular signal mediators and to control a large number of physiological and pathological processes, including cell proliferation and differentiation, the immune response, inflammation, cytoprotection and the febrile response. In particular, types PG-A and PG-J, which possess a cyclopentenonic structure, are strong inhibitors of virus replication [5]. Particularly, it has been recently demonstrated that cyclopentenonic prostaglandins inhibit HIV-1 virus replication, by blocking the viral RNAs transcription [6].

Prostaglandins are a group of hydrophobic compounds where modified fatty acids derivatives are attached to a 5-membered (cyclopentane) ring. They are found in many vertebrate tissues where they act as messengers involved in reproduction and in the inflammatory response to infection. Antiinflammatory substances inhibit prostaglandin synthesis, leading to reduced inflammation [7].

In 2002 new arachidonic acid peroxidation products were discovered. They are related biosynthetically to the isoprostanes and chemically characterized by a presence of a substituted tetrahydrofuran (oxacyclopentene) ring structure [8]. It was shown that they are produced in vivo by a free radical mechanism independent of the cyclooxygenase pathway. These substances seems to be involved in different processes, connected with inflammation development, lipid peroxidation, neurodegeneration, pulmonary function, oxidative stress, etc. Prostacyclin (Prostaglandin I₂) also contains conjugated oxacyclopentene cycle in molecule. Recently in U.S. Pat. No. 6,696,498 was described group of chemical substances, namely derivatives of 2-cyclopentene-1-one, with antiinflammatory, immunosuppressive, cytoprotective and antiviral activity. It was also claimed that the α,β-unsaturated carbonyl group in the cyclopentanone ring is the key structure necessary for such type of activity.

The main feature of these molecules was inhibitory action on inflammatory cascade, initiated by nuclear transcription factor, one of the early mediators of the immune and inflammatory responses. On the other hand, the effective concentration of these derivatives was high (up to 1000 mcmol).

New 2,3-dihydrofuran-2,3-dione derivatives were obtained by condensation of oxalyl chloride with Schiff bases of acetyl- or benzoylacetone and aromatic amines. These compounds showed only weak sedative action and weak analgesic effect [10].

In articles of Tolstikov G. et al. [11-14 ] synthetic methods for preparation of several derivatives of 2,3-dihydrofuranes are described. Revealed compounds showed some immune suppressory activity in vitro [11].

Kobayashi E. et al in U.S. Pat. No. 6,111,145 [15] described 4,5-dihydroxy-2-cyclopentene-1-one with anticancer and apoptosis inducing properties. Sets of benzimidazole and benzotriazole derivatives bearing on position 1 or 2 a tetrahydrofuranyl moieties were prepared through the addition of the suitable benzazoles on 2,3-dihydrofuran. Synthesized compounds were screened for in vitro antitumoral and anti-HIV-1 activities and most of them were found poorly active or completely inactive. On the other hand several compounds exhibited good tracheal relaxant activity in vitro; few compounds were more active than theophylline in this test. Another compound showed strong diuretic and saluretic activity [16].

A new class of synthetic biological response modifiers were produced by combining a highly electrophilic reactant 2-methyl-2,5-dihydrofuran with L-ascorbic acid. The chemical name of the product is 2-(5-methyl-2-furyl)-3-keto-L-butyrolactone. The substance is immunostimulatory in assays involving T- and B-lymphocytes [17]

### SUMMARY OF THE INVENTION

It has been found that many of 1-oxacyclopentene-2 derivatives possesses pronounced antiinflammatory, immunostimulatory, neuroprotective and cardiovascular activity. In a carrageenan paw edema model, investigated compounds showed activity higher than indomethacin or diclofenac, potent NSAlDs. Mechanism of such activity is unclear. It may be assotiated with interfering of the synthesis of such potent bioregulators as prostaglandins, prostanoids, prostacyclin, tromboxanes, leucotrienes and relative molecules, based on the arachidonic acid cascade [18]. 1-oxacyclopentene derivatives may trigger some mediator chains [19] or have another mechanism of action [20,21].

In one aspect the present invention provides a method for inhibiting an inflammatory response in a mammal comprising administrating a derivative of 1-oxacyclopentene-2. In one embodiment, the invention provides derivatives of 1-oxacyclopentene-2 that have pronounced anti-inflammatory activity in mammals.

In one aspect of the invention, it has been found that modification or substitution of the radicals attached to the oxalene-2 core of 1-oxacyclopentene-2 may change the intensity and duration of the anti-inflammatory activity.

In another aspect the present invention provides a use of 1-oxacyclopentene-2 derivatives as neuroprotectants, such as providing marked protection against brain injury, cerebral ischemia and stroke.

In another embodiment, the present invention provides the use of 1-oxacyclopentene-2 derivatives as modifiers of the immune system response of mammals.

Further embodiments of the invention are pharmaceutical compositions comprising 1-oxacyclopentene-2 derivatives for preparation of medicines for treatment of inflammation, neurodegeneration, autoimmune diseases, cerebral ischemia and neurotrauma, stroke, immunodeficiency diseases, pulmonary diseases, cardiovascular diseases, oncological diseases, infection diseases (bacterial, viral, fungal, parasitic and like), sepsis and septic shock.

Additional aspects and advantages of the present invention will be apparent in view of the description which follows. It should be understood, however, that, the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The invention will now be described in relation to the drawings, in which;

FIG. 1 shows the antiinflammatory action of 2-methyl-3-ethoxycatbonyl-4-isovaleryl-5-methoxy-(1-oxacyclopentene-2) (Example 1).

FIG. 2 shows the antiinflammatory action of 2-methyl-3-ethoxycarbonyl-4(n-hexyl)-5-methoxy-(1-oxacyclopentene-2) (Example 2).

FIG. 3 shows the antiinflammatory action of 2-methyl-3ethoxycarbonyl-4-(n-decyl)-5methoxy-(1-oxacyclopentene-2), (Example 3), delivered orally and parenterally.

FIG. 4 shows comparative antiinflammatory activity of two isomers of 2-methyl-3-ethoxycarbonyl-4(n-isovaleryl)-5-methoxy-(1-oxacyclopentene-2).

FIG. 5 shows the antiinflammatory activity of 2-methyl-3-ethoxycarbonyl-4-(2-methoxyphenyl)-5-methoxy-(1-oxacyclopentene-2) (Example 5) in different doses.

FIG. 6 shows the antiinflammatory activity of 1-oxacyclopentene-2 derivatives (Examples 5, 6).

FIG. 7 shows the recovery of immune response in immunosuppression, caused by cyclophosphamide.

FIG. 8 shows the reparation of humoral immune response after adriamycin-induced immunosuppression.

### DETAILED DESCRIPTION OF THE INVENTION

### Substituted Oxacyclopentene-2 Compounds

In one embodiment, the invention provides a substituted 1-oxacyclopentene-2 compound of the formula:

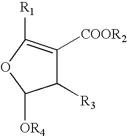

wherein R₁ is H, CH₃, CH₂X, CHXY,CXYZ (X,Y,Z are independently Cl, Br or F); alkyl, halogenated alkyl, alkenyl, cycloalkyl, cycloalkenyl, alkylaryl, aryl, substituted alkyl, substituted aryl, substituted alkylaryl;

R₂ is hydrogen, C1-C16 alkyl; cycloalkyl, cycloalkenyl, alkylaryl, aryl, substituted alkyl, substituted aryl, substituted alkylaryl;

R₃ is hydrogen, alkyl of 1-26 carbon atoms, (CH₂) ₘCH═CH—(CH₂)_{H}CH₃ (CH₂)ₘCH ═CH—(CH₂)ₙCH₃; (CH₂)ₘCH═CH—(CH₂)ₙCOOH; aryl, arylalkyl with 7-10 carbon atoms, wherein the aryl moiety of the aryl and arylalkyl groups is selected from the group consisting of phenyl, benzyl, naphthyl, pyridyl, quinolyl, isoquinolyl, quinoxalyl, thienyl, thionaphthyl, furyl, benzofuryl, benzodioxyl, benzoxazolyl, benzoisoxazolyl, and benzodioxolyl, and aryl may be optionally partially hydrogenated or substituted with a group selected from alkyl of 1-6 carbon atoms, arylalkyl of 7-10 carbon atoms, alkoxy of 1-6 carbon atoms, cyano, halogen, nitro, carbalkoxy of 2-7 carbon atoms alkyl chain, trifluoromethyl, amino, alkylamino or dialkylamino of 1-6 carbon atoms per alkyl group, alkylthio of 1-6 carbon atoms, —SO₃H, —PO₃H, and —COOH; m is 0-8; n is 1-8;

R₄ is hydrogen, CH₃, C₂H₅, alkyl of 3-16 carbon atoms, substituted alkyl, aryl, substituted aryl, alkylaryl or substituted alkylaryl groups which may be optionally partially or fully hydrogenated or substituted with a group selected from alkyl of 1-6 carbon atoms, arylalkyl of 7-10 carbon atoms, cyano, halogen, nitro, carbalkoxy of 2-7 carbon atoms alkyl chain, trifluoromethyl, amino, alkylamino or dialkylamino of 1-6 carbon atoms per alkyl group;

and any diastereromer, physiological or pharmaceutical acceptable salt thereof, addition salts and esters thereof. Compositions comprising same, such as pharmaceutical compositions comprising said compounds and a pharmaceutical carrier are provided.

### Pharmaceutical Compositions and Modes of Administration

“Effective Amount” and “Therapeutically Effective Amount” as used herein means an amount effective, at dosages and for periods of time necessary to achieve the desired results. For example, an effective amount of a substance may vary according to factors such as the disease state, age, sex, and weight of the individual, and the ability of the substance to elicit a desired response in the individual. Dosage regimes may be adjusted to provide the optimum therapeutic response. For example, several divided doses may be administered daily or the dose may be proportionally reduced as indicated by the exigencies of the therapeutic situation.

Substituted oxacyclopentene-2 or a derivative thereof, such as 1-oxacyclopentene-2-derivatives of the invention, pharmaceutically acceptable salts, addition salts or esters thereof, or any diastereomer thereof or obvious chemical equivalents thereof can be administered by any means that produce contact of said active agent with the agent's sites of action in the body of a subject or patient to produce a therapeutic effect, in particular a beneficial effect, in particular a sustained beneficial effect. The active ingredients can be administered simultaneously or sequentially and in any order at different points in time to provide the desired beneficial effects. A compound and composition of the invention can be formulated for sustained release, for delivery locally or systemically. It lies with the capability of a skilled physician or veterinarian to select a form and route of administration that optimizes the effects of the compositions and treatments of the present invention to provide therapeutic effects, in particular beneficial effects, more particularly sustained beneficial effects.

In one embodiment, administration of substituted oxacyclopentene-2 or a derivative thereof, such as 1-oxacyclopentene-2-derivatives of the invention, pharmaceutically acceptable salts, addition salts or esters thereof or diastereomers thereof or obvious chemical equivalents thereof includes any mode that produce contact of said active agent with the agent's sites of action in vitro or in the body of a subject or patient to produce the desired or therapeutic effect, as the case may be. As such it includes administration of the compound(s) to the site of action—directly or through a mode of delivery (e.g. sustained release formulations, delivery vehicles that result in site directed delivery of the peptide to a particular cell or site in the body.

The above described substances including substituted oxacyclopentene-2 or a derivative thereof, such as 1-oxacyclopentene-2-derivatives of the invention, pharmaceutically acceptable salts, addition salts or esters thereof or diastereomer thereof, or obvious chemical equivalents thereof may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals.

Thus in one embodiment, the invention provides the use of substituted oxacyclopentene-2 or a derivative thereof, such as 1-oxacyclopentene-2-derivatives of the invention, pharmaceutically acceptable salts, addition salts or esters thereof or diastereomers thereof, or obvious chemical equivalents thereof for the treatment of pulmonary disease and/or related conditions; cardiovascular disease and/or related condition; allergy and/or related conditions; oncological disease and/or related conditions; infection or infectious diseases (e.g. bacterial, viral, fungal, parasitic and the like) and/or related conditions; sepsis, septic shock and/or related conditions. In one embodiment, a therapeutically effective amount of substituted oxacyclopentene-2 or a derivative thereof, such as 1-oxacyclopentene-2-derivatives of the invention, pharmaceutically acceptable salts, addition salts or esters thereof or diastereomers thereof, or obvious chemical equivalents thereof or a pharmaceutical composition as described herein is administered to a patient in need thereof. A patient in need thereof is any animal, in one embodiment a human, that may benefit from substituted oxacyclopentene-2 or a derivative thereof, such as 1-oxacyclopentene-2-derivatives of the invention, pharmaceutically acceptable salts, addition salts or esters thereof or diastereomers thereof or obvious chemical equivalents thereof and its effect on the conditions noted herein above.

An active substance may be administered in a convenient manner such as by injection (subcutaneous, intravenous, etc.), parentreral, oral administration, topical, inhalation, transdermal application, recta, transmucosal, ocular, intranasal, sublingual, intradermal, intracranial, intrathecal, or intralumbar administration. Depending on the route of administration, the active substance may be coated in a material to protect the compound from the action of enzymes, acids and other natural conditions that may inactivate the compound. In one embodiment, substituted oxacyclopentene-2 or a derivative thereof, such as 1-oxacyclopentene-2-derivatives of the invention, pharmaceutically acceptable salts, addition salts or esters thereof or diastereomers thereof or obvious chemical equivalents thereof is administered directly to or proximate to the desired site of action, by injection or by intravenous.

The compounds and compositions described herein can be prepared by per se known methods for the preparation of pharmaceutical acceptable compositions which can be administered to subjects, such that an effective quantity of the active substance is combined in a mixture with a pharmaceutical acceptable vehicle or carrier. Suitable vehicles or carriers are described, for example, in Remington's Pharmaceutical Sciences (Remington's Pharmaceutical Sciences, Mack Publishing Company, Easton, Pa., USA 1985 or Remington's The Sciences and Practice of Pharmacy, 21st Edition”, (University of the Sciences in Philadelphia, 2005) or Handbook of Pharmaceutical Additives (compiled by Michael and Irene Ash, Gower Publishing Limited, Aldershot, England (1995)). On this basis, the compositions include, albeit not exclusively, solutions of the substances in association with one or more pharmaceutical acceptable vehicles, carriers or diluents, and may be contained in buffered solutions with a suitable pH and/or be iso-osmotic with physiological fluids. In this regard, reference can be made to U.S. Pat. No. 5,843,456.

As will also be appreciated by those skilled, administration of substances described herein may be by an inactive viral carrier. In one embodiment substituted oxacyclopentene-2 or a derivative thereof such as 1-oxacyclopentene-2-derivatives of the invention, pharmaceutically acceptable salts, addition salts or esters thereof or diastereomers thereof or obvious chemical equivalents thereof can be administered in a vehicle comprising saline and acetic acid.

In one embodiment, the substituted oxacyclopentene-2 or a derivative thereof, such as 1-oxacyclopentene-2-derivatives of the invention, pharmaceutically acceptable salts, addition salts or esters thereof or diastereomers thereof or obvious chemical equivalents thereof are formulated into 0.001-50 mg/kg dosage forms. In another embodiment, they are administered in a daily dosage form of 0.001-50 mg/kg.

### EXAMPLES

1-oxacyclopentene-2 derivatives were synthesized according to methods, described in [Khimiko-Pharmacevticheskii Zhurnal, 1989, #6, pp. 672-675].

Preparation of Oxacyclopentene-2 Derivatives (modified from Khimiko-Pharmacevticheskii Zhurnal, 1989, #6, pp. 672-675).

Step 1. Interaction of equimolar amounts of Acetylacetonate* and corresponding aldehyde was initiated by addition of a basic catalyst (e.g., freshly distilled piperidine) in alcoholic solution. Reaction was stopped by pouring into a mixture of ice and water. Condensation products (mixture of corresponding enones) were extracted with diethyl ether, washed with water, dried and solvent was evaporated at low pressure. (*Acetylacetonate can be replaced by another Michael's condensation product, e.g., adduct of carboxylic acid ester with corresponding ketone).

Step 2. Obtained in step 1 product was treated with methanolic solution of equimolar amount of purified nitromethane in presence of anhydrous sodium methylate. Reaction progress was controlled by TLC. Reaction mixture was treated with excess of methanolic solution of anhydrous sulfuric acid.

Step 3. Reaction mixture of step 2 was diluted with water and extracted with organic solvent (methylene chloride, chlorophorm, ethylacetate, hexane, diethyl ether). Organic layer was washed with water and dried with anhydrous magnesium sulphate. Organic solvent was evaporated at low pressure.

Step 4. Residue from step 3 was dissolved in toluene and treated with catalytic amount of p-toluenesulfonic acid under TLC control or until desired level of cleavage achieved.

Step 5. Product was purified using column chromatography (Alumina, Brockman activity I, or Kieselgur/Silicagel, 80-100 mesh, Sigma-Aldrich) with appropriate eluent (mixture of hexane-ethyl acetate, hexane-ethanol or hexane-ethyl ether).

Partial list of synthesized compounds (for illustrative purposes, but not limiting) is presented in Table 1.

According to present invention, 1-oxacyclopentene-2 derivatives are able to suppress inflammation, provide cardiovascular, neuroprotective and immune stimulation activity in mammals in doses 0.01-50 mg/kg.

Anti-Inflammatory Activity of Substituted 1-oxacyclopentene-2 Derivatives

Anti-inflammatory activity was investigated using Carrageenan Paw Edema Model in rats [22]

### Lambda-Carrageenan Induced Paw Edema in Rats

Animals are housed at the in air-conditioned facility at 22±1° C. and constant humidity. Standard diet and water were available ad libitum.

Before injection of carrageenan, rats are anesthetized with Xylosin (5 mg/kg) and Ketamine (40 mg/kg) combination, injected intraperitoneally as solution in sterile saline. Fifteen minutes later one paw is injected with 0.1 ml of 1% solution of λ-carrageenan. Carrageenan have to be prepared as 1% solution in sterile saline 24 h before an experiment by continuous stirring with heating at 60° C. on magnetic stirrer (1-1.5 hours), followed by sterile filtration through 0.22 mcm Acrodisc polysulphone membrane filter in aseptic conditions.

An appropriate amount of investigated compound of this invention, dissolved in vehicle, comprised of 10% EtOH, 40% PEG-400, 5% Tween-80 and 45% of water for injection (all used materials USP/NF grade). Obtained solution was administered to Male Wistar rats (200-250 g), vehicle was used as placebo control. The volume of the administered vehicle for a compound of this invention was controlled to be no more than 1-2 ml/kg body weight of the animal. In order to determine the inhibitory activity of a compound of this invention at a dose, usually 6 to 10 animals were used both for drug-treated group and for control group.

This protocol prevented the appearance of behavioural signs of pain during and after the injections.

Each animal is marked at Tibia-Calcaneus joint of the hind paw using waterproof marker. Testing formulation are administrated 30 minutes before Carrageenan injection in case of systemic delivery (oral, parenteral etc.). The volume of the carrageenan-injected and contralateral saline-injected paw was measured using a plethysmometer Model 7141 (Ugo Basile, Varese, Italy). The plethysmometer was calibrated and tested according to manufacturer's Manual.

Intraplantar subcutaneous injections are made with a insulin syringe (0.5 ml) and a 30-gauge hypodermic needle in volumes of 100 mcl. The needle is inserted into the pad region of the glabrous skin and moved 5±1 mm proximal towards the tarsal region. The carrageenan solution is injected slowly in a warm state (˜37° C.) in volume of 100 μl. Foot volumes are measured immediately before the injection of carrageenan and saline and at different intervals thereafter (1 h, 2 h, 3 h, 4 h, 5 h and 6 h). The paw is dipped into a cylinder filled with water (contain 3 ml/l of wetting compound and 0.5 g/l of Sodium chloride), exactly up to the reference mark on the paw. The difference (mcl) in volume between the right paw (carrageenan-injected) and left paw (saline-injected) is calculated (Δ) and used as estimate of edema.

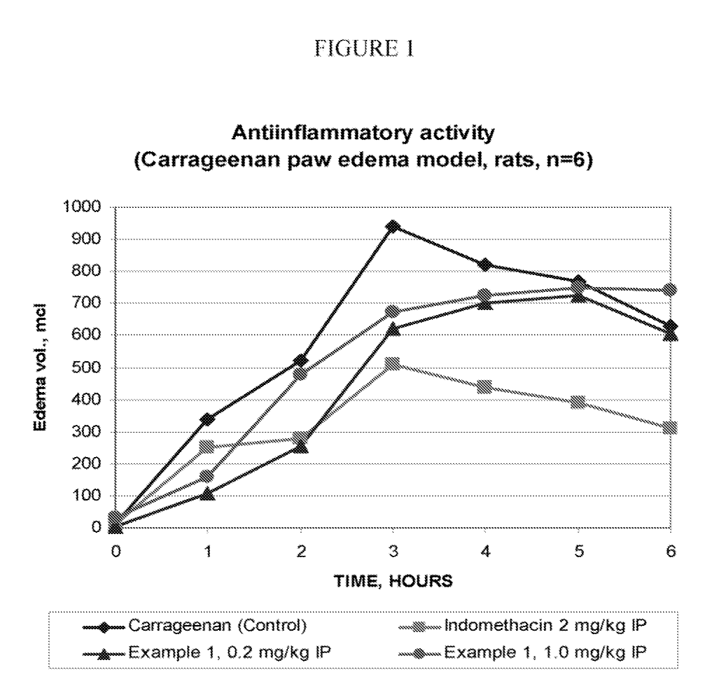

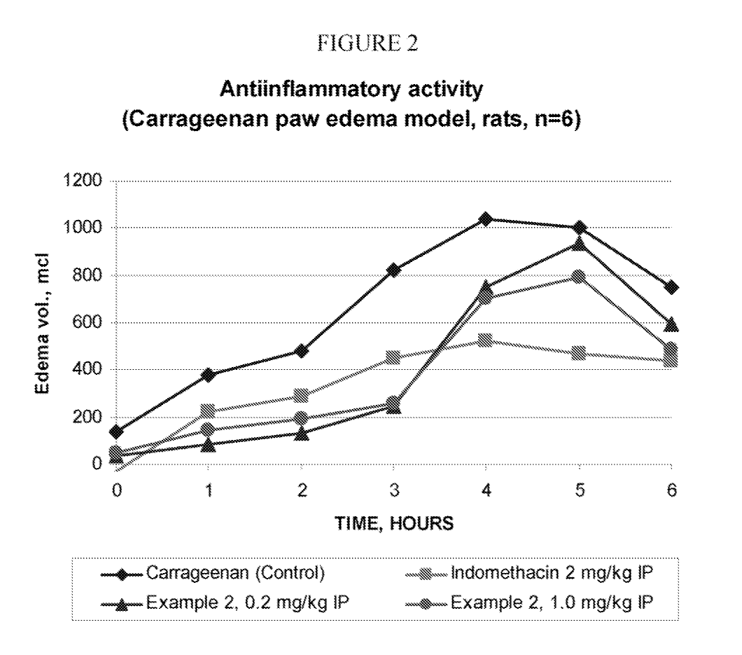

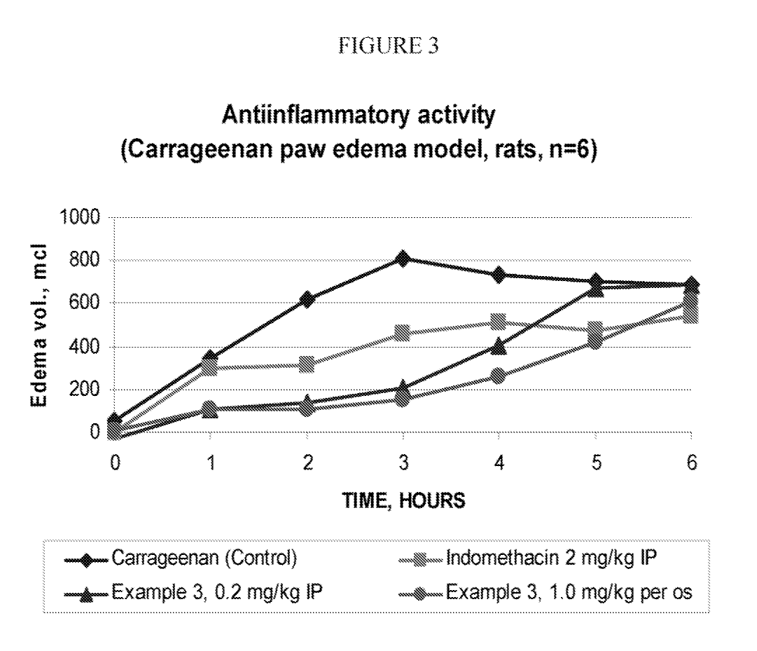

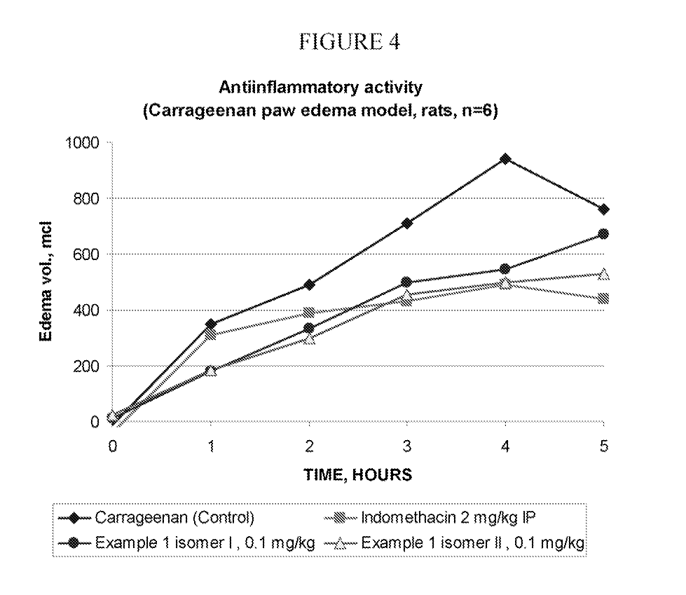

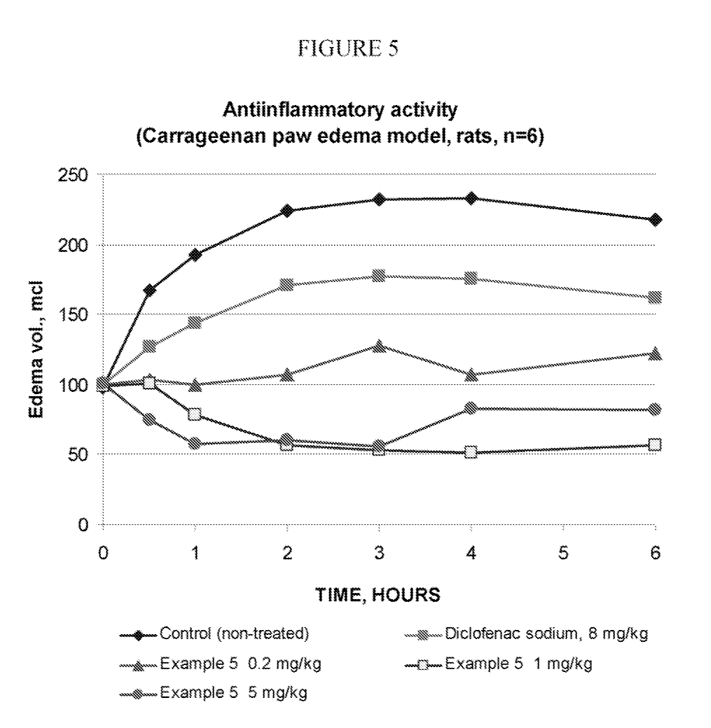

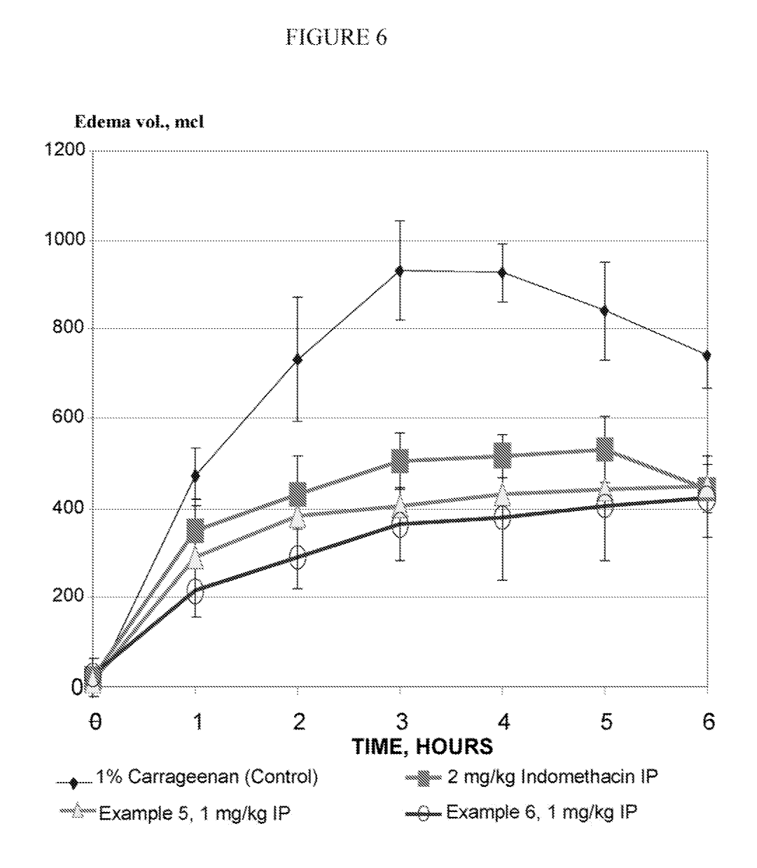

Carrageenan Foot Edema (CFE) inhibition data for selected compounds of this invention are summarized in the following FIGS. 1-6.

### Immunomodulatory Activity of Substituted 1-Oxacyclo-Pentene-2 Derivatives

Immunomodulation has been of great interest for possible application in the treatment of diseases such as AIDS, various neoplasms, a variety of severe viral, bacterial and fungal infections, as well as skin and joints disorders. Very few known substances demonstrate immunostimulatory activity. Natural compounds and extracts, such as Panax ginseng and relatives, Ganoderma and other mushrooms extracts, plant and yeast polyglucans, and some others, show non-reproducible and ambiguous results. Peptides and proteins (interferons, interleukins, antibodies and vaccines) are difficult to manufacture and purify, expensive and demonstrate uncertain activity and many cause undesirable side effects.

Some of 1-oxacyclopentene-2 derivatives were investigated for immunomodulatory activity. The most active of the tested compounds demonstrated a noticeable stimulation of both humoral (antibodies production) and cellular immune response (T-cells response) in mice.

Immune response caused by substituted 1-oxacyclopentene-2 derivatives was evaluated using Plaque Forming Cell assay (PFC) and Delayed Type Hypersensitivity (DTH) analysis.

### Plaque-Forming Cell Assay (PFC)

Humoral-mediated immune response is routinely measured using the antibody-forming cell (AFC) assay. Primarily used for evaluating humoral immunity, this assay, in fact, evaluates overall immune competence including T- and B-cell function, antigen processing and presentation. Sheep Red Blood Cells (SRBC) are the most common T-dependent antigen used for this assay.

Primary IgM response after SRBC administration was quantitatively estimated using a modified hemolytic plaque-forming cell assay [23-25]

Since SRBC are T-dependent antigen, a normal AFC response depends on multiple factors: antigen processing and presentation actuated by macrophages, appropriate T-cell signalling, B-cells differentiation, and affinity maturation of antibodies in plasma cells.

For induction of a primary humoral PFC response, C57BL/6J mice were immunized intraperitoneally with 0.1 ml of a 5% suspension of SRBC, containing 1×10⁸ cells. On day 5 after immunization animals were sacrificed under deep ether anesthesia, spleens were dissected out and minced through a stainless steel mesh. Individual spleen cell suspensions were washed three times with Hank's BSS, nucleated cells number was determined with a haemocytometer, viability was assessed by exclusion of trypan blue and adjusted to 1×10⁷ viable cells per ml in Medium 199 containing Earle's salts and L-glutamine.

For the PFC assay, suspensions containing 1×10⁶ spleen cells, 1×10⁹ SRBCs and guinea pig serum (diluted 1:10, a source of complement), were introduced into a Cunningham chambers (100 μl volume) and incubated for 1 h at 37° C. The number of hemolytic plaques, produced by anti-SRBC IgM secreting plasma cells, was counted using a light microscope, and the number of PFC determined for each mice was expressed per 1×10⁶ spleen cells and per whole spleen.

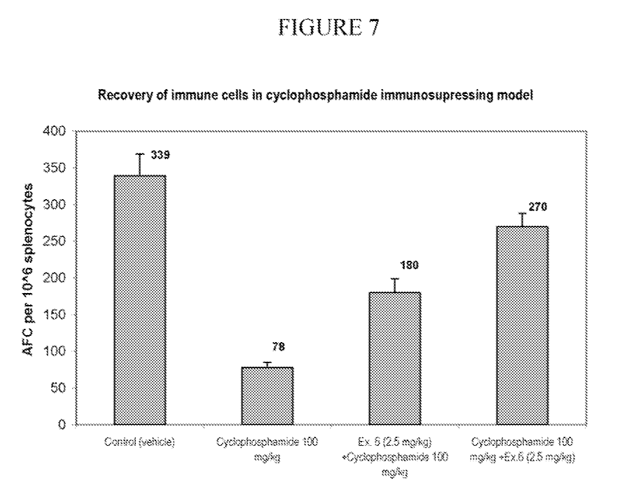

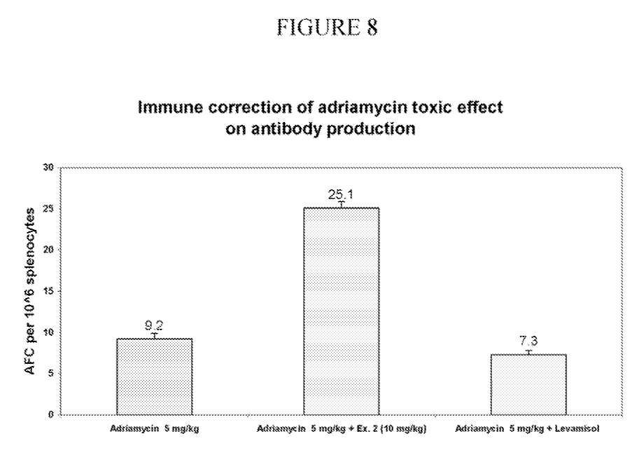

Results of immunomodulatory activity of selected compounds of this invention are represented in the Table 1 and FIG. 7 and FIG. 8.

Antibodies formation in spleen and lymph nodes increased 300-500% (ex. 1, 6, 10) as compared to control groups; administrated doses were in range 2.5-10 mg/kg (approximately 0.5-1% of LD₅₀). By comparison, Levamisol, used as positive reference, showed 20% increase in antibody formation at doses of 2.5 mg/kg (10% of LD₅₀).

Cellular immune response was increased 2-3.5 times for some of the compounds (ex. 3, 9, 10), compared to a 240% increase after Levamisol administration.

Results obtained in recovery from induced immunosupression, are presented at FIG. 7 and FIG. 8. After significant suppression of immunity induced by gamma-irradiation or cytostatic drugs, oxacyclopentene-2 derivatives showed pronounced immunoprotective (when administered before damaging factor) and immunocorrective (after developed damage) effects.

### Delayed Type Hypersensitivity Test (DTH)

The method of Lagrange et al. [26] was used. Mice were sensitized intravenously (iv) with 10⁵ SRBC in saline. After 4 days, the mice were given eliciting dose of the antigen: 10⁸ SRBS in 20 mcl of sterile saline into right hind paw, left hind paw was injected with 20 mcl of sterile saline. In 24 hours after challenge, the foot pad edema volume was measured using a caliper. The difference in volume (mcl) between the right paw (antigen-injected) and left paw (saline-injected) is calculated (Δ) and used as edema volume.

### Immunosuppression Models

Immunosupression in mice was induced by parentreral administration of Adriamycin or Cyclophosphamide. Mice were injected with Adriamycin intraperitoneally in a dose of 5 mg/kg one day before the immunization with SRBC. Cyclophosphamide was used in a dose of 100 mg/kg 24 hours before the antigen challenge with SRBC.

One of the most potent immunosupressive agents, Adriamycin, almost completely destroys antibody production after its administration, and total number of AFC drops to 1-2% of initial values. Even in this extreme situation, administration of 2-methyl-3-ethoxycarbonyl-4-(2-methoxyphenyl)-5-methoxy-(1-oxacyclopentene-2, example 6) in dose 2.5 mg/kg produces a nearly 3 folds increase in antibody levels. Levamisol demonstrates no antibody response in this type of immunosuppression model. (See FIG. 8).

While the present invention has been described with reference to what is presently considered to be a preferred embodiment, it is to be understood that the invention is not limited to the disclosed embodiment. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Derivatives of 1-oxacyclopentene-2 and immunomodulatory properties thereof

| | | | | | | | | DHT, |
|---|---|---|---|---|---|---|---|---|
| | | | | | AFC number increase, | | | % of |
| | | | | | % of control | | | control |
| Ex | R1 | R2 | R3 | R4 | 2.5 mg/kg | 5 mg/kg | 10 mg/kg | 10 mg/kg |
| 1 | CH3 | COOEt | isobutyl | OCH3 | 209% | 275% | 453% | 300% |
| 2 | CH3 | COOEt | n-pentyl | OCH3 | 360% | 331% | 330% | 220% |
| 3 | CH3 | COOEt | n-nonyl | OCH3 | 225% | 289% | 326% | 250% |
| 4 | CH3 | COOEt | n-undecyl | OCH3 | 253% | 330% | 282% | 240% |
| 5 | CH3 | COOEt | phenyl | OCH3 | 202% | 324% | 259% | 200% |
| 6 | CH3 | COOEt | 2-methoxyphenyl | OCH3 | 260% | 229% | 440% | 190% |
| 7 | CH3 | COOEt | 2,4-dichlorophenyl | OCH3 | 269% | 302% | 358% | 230% |
| 8 | CHBr₂ | COOEt | phenyl | OCH3 | 188% | 276% | 271% | 260% |
| 9 | CH3 | COOH | isobutyl | OCH3 | 230% | 320% | 360% | 360% |
| 10 | CH3 | COO⁺Li⁻ | isobutyl | OCH3 | 290% | 320% | 470% | 360% |
| 11 | CH3 | COOEt | n-hexyl | OCH3 | 245% | 241% | 287% | 210% |
| 12 | CH3 | COOEt | n-butyl | OCH3 | 251% | 239% | 291% | 230% |
| 13 | CH3 | COOEt | 3-pyridil | OCH3 | 168% | 201% | 191% | 180% |
| 14 | CH3 | COOEt | 3-methoxyphenyl | OCH3 | 135% | 187% | 254% | 200% |
| 15 | CH3 | COOEt | 4-methoxyphenyl | OCH3 | 291% | 312% | 267% | 160% |
| 16 | C2H5 | COOEt | 2,4-dimethylphenyl | OCH3 | 165% | 192% | 217% | 140% |
| 17 | C2H5 | COOEt | (CH2)2═(CH2)2COOH | OCH3 | 129% | 143% | 111% | 170% |
| 18 | CH3 | COOH | (CH2)2═(CH2)2COOEt | OC2H5 | 138% | 122% | 154% | 190% |

| | | | |
|---|---|---|---|
| Levamisol (positive control, immunostimulator), dose | 121% | ND (toxic effects) | 240% |
| 2.5 mg/kg | | | |

### REFERENCES

1. U.S. Pat. No. 5,710,289 Falling S N, et al. Process for the preparation of dihalotetrahydrofurans from dihydrofuran.
2. Miles W H, Dethoff E A, Tuson H H, Ulas G J Kishner's “Reduction of 2-furylhydrazone gives 2-methylene-2,3-dihydrofuran, a highly reactive ene in the ene reaction.” Org Chem. 2005 April 1;70(7):2862-5.
3. Harris E. G, et al. U.S. Pat. No. 4,681,703 “Alkyl substituted-2,3-dihydrofuran fragrance compositions”.
4. Harris E. G., et al., U.S. Pat. No. 4,515,978 “Dihydrofurans”.
5. “Stress Proteins: Induction and Function” Editors Schlesinger M J, Garaci E., Santoro M. G., Springer-Verlag, Heidelberg-Berlin, 1990, p. 2744.
6. C. Rozera et al., Inhibition of HIV-1replication by cyclopentanone prostaglandins in acutely infected human cells. Evidence for a transcriptional block, J. Clin. Invest. 97: 1795-996.
7. S. Narumiya, Y. Sugimoto, F. Ushikubi. Physiological Reviews, Vol. 79, No. 4, Oct. 1999, pp. 1193-122.
8. J. P. Fessel et al., Discovery of lipid peroxidation products formed in vivo with a substituted tetrahydrofuran ring (isofurans) that are favored by increased oxygen tension. Proc Natl Acad Sci U S A. 2002 Dec. 24; 99(26): 16713-8.
9. U.S. Pat. No. 6,696,498 (Santoro et al.).
10. W. Zankowska-Jasinska, et al., Synthesis and pharmacological activity of some 2,3-dihydrofuran-2,3-dione derivatives. Pol J Pharmacol Pharm. 1982, 34(5-6), pp. 391-398.
11. Tolstikov G. et al., Prostanoids. Khimiko-Pharmacevticheskii Zhurnal, 1989, Vol. 25, No. 6, pp. 672-675.
12. Tolstikov G. et al., trans-2-Methoxy-4-hydroxymethyl-3,5-dimethyl-2,3-dihydrofuran as biological isoster of botryodiplodin. Zhurnal Organicheskoi Khimii, 1988, vol. 24, No. 12, pp. 2626-2627.
13. Tolstikov G. et al., The cyclic analogs of levuglandins. Zhurnal Organicheskoi Khimii, 1990, vol. 26, No. 1, pp. 119-127.
14. Tolstikov G. et al., The cyclic analogs of levuglandins. Zhurnal Organicheskoi Khimii, 1990, vol. 26, No. 10, pp. 2145-2156
15. Kobayashi E. et al., U.S. Pat. No. 6,111,145 Cyclopentanone derivative described 4,5-dihydroxy-2-cyclopentene-1-one with anticancer and apoptosis inducing properties.
16. Sparatore A. et al., Synthesis and biological investigations of 1-(tetrahydropyran-2-yl)-and 1 -(tetrahydrofuran 2′-yl)benzimidazoles and ½-(tetrahydropyran-2′-yl) and ½-(tetrahydrofuran-2′-yl)benzotriazoles. II Farmaco. 1997 Aug.-Sept.; 52(8-9): 509-521.
17. Veltri R. W. et al., A new class of synthetic biological response modifiers: the methylfurylbutyrolactones (Nafocare B). J Biol Response Mod. 1986 No. 5 (5) pp. 444-461.
18. Arachidonic acid and free fatty acids as second messengers and the role of protein kinase C. Khan W A; Blobe G C; Hannun Y A, Cell Signal 1995 Mar.;7(3): 171-84.
19. H. Beker et al., Clin. Exp. Immunol. 99; 325, 1995.
20. Rosenkranz B. et al., Effects of salicylic and acetylsalicylic acid alone and in combination on platelet aggregation and prostanoid synthesis in man. Br J Clin Pharmacol 1986 Vol. 21 No. 3 pp. 309-317.
21. Abramson S. B., Weissmann G. The mechanisms of action of nonsteroidal antiinflammatory drugs. Arthritis Rheum 1989 Vol 32 No. 1 pp. 1-9.
22. Winter C. A., et al., Proc. Soc. Exp. Biol. Med, 111 (1962), pp. 544-547. Detailed description of the method was obtained in “Current protocols in Pharmacology, (J. Wiley & Sons, 1998), u. 5.4.1-5.4.3.
23. Jerne, N. K. and Nordin, A. A. Plaque formation in agar by single antibody-producing cells. Science. 1963; 140, 405-406.
24. Cunningham, A. J. A method of increased sensitivity for detecting single antibody-forming cells. Nature. 1965; 207(1): 1106-7.
25. Cunningham A J. and Szenberg A. Further improvements in the plaque technique for detecting single antibody-forming cells. Immunology. 1968: Vol. 14 No. 4 pp. 599-600.
26. Lagrange P. H. et al., Influence of dose and route of antigen injection on the immunological induction of T cells. J Exp Med. 1974; Vol. 139 No. 3 pp. 528-542.

1. A method of treating inflammatory status and inflammation-related conditions in mammals, comprising administering to a subject in which such treatment is required a substituted 1-oxacyclopentene-2 or a pharmaceutically acceptable derivative thereof in a daily dosage of 0.001-50 mg/kg.

2. A method of treating neurodegeneration and neurodegeneration related conditions in mammals, comprising administering to a subject in which such treatment is required a substituted oxacyclopentene-2 or a pharmaceutically acceptable derivative thereof in a daily dosage of 0.001-50 mg/kg.

3. A method of treating autoimmune diseases and autoimmune diseases related conditions in mammals, comprising administering to a subject in which such treatment is required a substituted oxacyclopentene-2 or a pharmaceutically acceptable derivative thereof in a daily dosage of 0.001-50 mg/kg.

4. A method of treating cerebral ischemia and cerebral ischemia related conditions in mammals, comprising administering to a subject in which such treatment is required a substituted oxacyclopentene-2 or a pharmaceutically acceptable derivative thereof in a daily dosage of 0.001-50 mg/kg.

5. A method of treating neurotrauma and neurotrauma related conditions in mammals, comprising administering to a subject in which such treatment is required a substituted oxacyclopentene-2 or a pharmaceutically acceptable derivative thereof in a daily dosage of 0.001-50 mg/kg.

6. A method of treating immunodeficiency diseases and immunodeficiency diseases related conditions in mammals, comprising administering to a subject in which such treatment is required a substituted oxacyclopentene-2 or a pharmaceutically acceptable derivative thereof in a daily dosage of 0.001-50 mg/kg.

7. A method of treating pulmonary diseases and pulmonary diseases related conditions in mammals, comprising administering to a subject in which such treatment is required a substituted oxacyclopentene-2 or a pharmaceutically acceptable derivative thereof in a daily dosage of 0.001-50 mg/kg.

8. A method of treating cardiovascular diseases and cardiovascular diseases related conditions in mammals, comprising administering to a subject in which such treatment is required a substituted oxacyclopentene-2 or a pharmaceutically acceptable derivative thereof in a daily dosage of 0.001-50 mg/kg.

9. A method of treating allergy and allergy related conditions in mammals, comprising administering to a subject in which such treatment is required a substituted oxacyclopentene-2 or a pharmaceutically acceptable derivative thereof in a daily dosage of 0.001-50 mg/kg.

10. A method of treating oncological diseases and oncological diseases related conditions in mammals, comprising administering to a subject in which such treatment is required a substituted oxacyclopentene-2 or a pharmaceutically acceptable derivative thereof in a daily dosage of 0.001-50 mg/kg.

11. A method of treating infection diseases (bacterial, viral, fungal, parasitic and like) and infection diseases related conditions in mammals, comprising administering to a subject in which such treatment is required a substituted oxacyclopentene-2 or a pharmaceutically acceptable derivative thereof in a daily dosage of 0.001-50 mg/kg.

12. A method of treating sepsis and septic shock and sepsis related conditions in mammals, comprising administering to a subject in which such treatment is required a substituted oxacyclopentene-2 or a pharmaceutically acceptable derivative thereof in a daily dosage of 0.001-50 mg/kg.

13. A substituted 1-oxacyclopentene-2 compound of the formula: 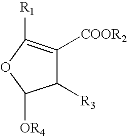
wherein R₁ is H, CH₃, CH₂X, CHXY, CXYZ (X,Y,Z are independently Cl, Br or F); alkyl, halogenated alkyl, alkenyl, cycloalkyl, cycloalkenyl, alkylaryl, aryl, substituted alkyl, substituted aryl, substituted alkylaryl;
R₂ is hydrogen, C1-C16 alkyl; cycloalkyl, cycloalkenyl, alkylaryl, aryl, substituted alkyl, substituted aryl, substituted alkylaryl;
R₃ is hydrogen, alkyl of 1-26 carbon atoms, (CH₂)ₘCH═CH—(CH₂)ₙCH₃(CH₂) ₘCH═CH—(CH ₂)ₙCH₃; (CH₂)ₘCH═CH—(CH₂)ₘCH═CH—(CH₂)ₙCOOH; aryl, arylalkyl with 7-10 carbon atoms, wherein the aryl moiety of the aryl and arylalkyl groups is selected from the group consisting of phenyl, benzyl, naphthyl, pyridyl, quinolyl, isoquinolyl, quinoxalyl, thienyl, thionaphthyl, furyl, benzofuryl, benzodioxyl, benzoxazolyl, benzoisoxazolyl, and benzodioxolyl, and aryl may be optionally partially hydrogenated or substituted with a group selected from alkyl of 1-6 carbon atoms, arylalkyl of 7-10 carbon atoms, alkoxy of 1-6 carbon atoms, cyano, halogen, nitro, carbalkoxy of 2-7 carbon atoms alkyl chain, trifluoromethyl, amino, alkylamino or dialkylamino of 1-6 carbon atoms per alkyl group, alkylthio of 1-6 carbon atoms, —SO₃H,—PO₃H, and —COOH; m is 0-8; n is 1-8;
R₄ is hydrogen, CH₃, C₂H₅, alkyl of 3-16 carbon atoms, substituted alkyl, aryl, substituted aryl, alkylaryl or substituted alkylaryl groups which may be optionally partially or fully hydrogenated or substituted with a group selected from alkyl of 1-6 carbon atoms, arylalkyl of 7-10 carbon atoms, cyano, halogen, nitro, carbalkoxy of 2-7 carbon atoms alkyl chain, trifluoromethyl, amino, alkylamino or dialkylamino of 1-6 carbon atoms per alkyl group.

14. A pharmaceutical composition comprising a pharmaceutically effective amount of a compound of claim 13, any diastereomer of the compound or a pharmaceutically acceptable salt thereof and a pharmaceutically acceptable carrier.

15. A pharmaceutical composition of claim 14, intended for administration by oral, parenteral, inhalation, topical, transdermal, rectal, transmucosal, ocular, intranasal, sublingual, intradermal, intracranial, intrathecal, intralumbar ways.
